(19) United States
(12) Patent Application Publication
(10) Pub. No.: US 2011/0224511 A1
(43) Pub. Date: Sep. 15, 2011

(54) METHOD AND SYSTEM FOR MEASURING VITAL FUNCTIONS OF HUMAN BODY

(76) Inventors: Seppo Säynäjäkangas, Oulu (FI); Jari Räglund, Helsinki (FI)

(21) Appl. No.: 13/130,704

(22) PCT Filed: Nov. 20, 2009
(86) PCT No.: PCT/FI2009/050944

§ 371 Date: May 23, 2011

(30) Foreign Application Priority Data
Nov. 21, 2008 (FI) 20086107

(51) Int. Cl. A61B 5/00

(52) U.S. Cl. 600/301

(57) ABSTRACT

Method and system for measuring the activity of the functions of the body of a person, wherein the vital functions are measured wirelessly with a portable activity measuring apparatus. For measuring an activity a sensor or sensors, e.g. activity sensors, blood pressure sensors, ECG sensors or heart pulse sensors, that are fixable to the body arc used, from which sensor or sensors a measured value or measured values are received, e.g. activity values, blood pressure values, ECG curves or heart pulse values. The aforementioned value or aforementioned values are sent wirelessly to the server of the measuring of an activity for processing, monitoring, analysis and/or other actions. The server analyzes the measured values, converts the processed measurement results into a suitable format for sending onwards, and sends the converted measurement results to the user/users.

### FIELD OF THE INVENTION

The invention relates to a method and an apparatus for measuring the vital functions of the body of a person. More particularly, the invention relates to a method and an apparatus for measuring the vital functions of the body of a person with an activity meter to be fixed to the arm.

### PRIOR ART

In the measurement of an active person, as well as in rehabilitation and in the measurement of the vital functions of a patient, it is important to measure vital functions accurately without breaks. Different medical apparatuses are known in measurements of the bodily activities of a person, in which apparatuses an activity of a human body, heart functions—e.g. ECG activity, the pulse rate of a heart and blood pressure, among other things, are measured.

Fl patent publication 68734 also discloses a wireless telemetric measurement for measuring heart rate, wherein telemetric data transfer occurs using a magnetic near-field by controlling magnetic windings in the transmitter unit with a heart rate or ECG signal expressed from the body and amplified, the directional magnetic field produced by which windings is essentially multidimensional, and by expressing the aforementioned magnetic field in the receiver with a corresponding winding construction possessing the directional pattern of the multidimensional magnetic field.

The measurement of blood pressure can be performed e.g. with OMRON M4-1 pat. CE 0197 or other corresponding technology and blood pressure readings and also the heart pulse rate reading can be transferred telemetrically to an activity meter fixed to the arm.

The preceding describes the automatic measurement of an activity of movement functions of the body, of the ECG activity of the heart and of the pulse of the heart, and also describes the transfer to an activity meter kept on the arm. Correspondingly it is possible to measure and transfer many other medical measurements automatically to an activity meter of the body that is worn on the arm.

### SUMMARY OF THE INVENTION

The purpose of this invention is to achieve a new type of method and system for the continuous measurement of an activity of a human body.

The invention is a method and a system for wirelessly measuring an activity of a human body in such a way as is presented in detail in the independent claims below. The system according to the invention is extremely simple and operationally reliable, and also inexpensive in price.

In the solution of the invention a SEIKO CAL 5M62 watch can function as an automatic power source of an activity measuring device of the human body. The charging of the watch functions as a sensor device that is kept on the wrist of a person and that stores the kinetic energy of the wrist movement of the person. The voltage received from the device can be transferred to an activity meter fixed to the arm.

### SHORT DESCRIPTION OF THE DRAWINGS

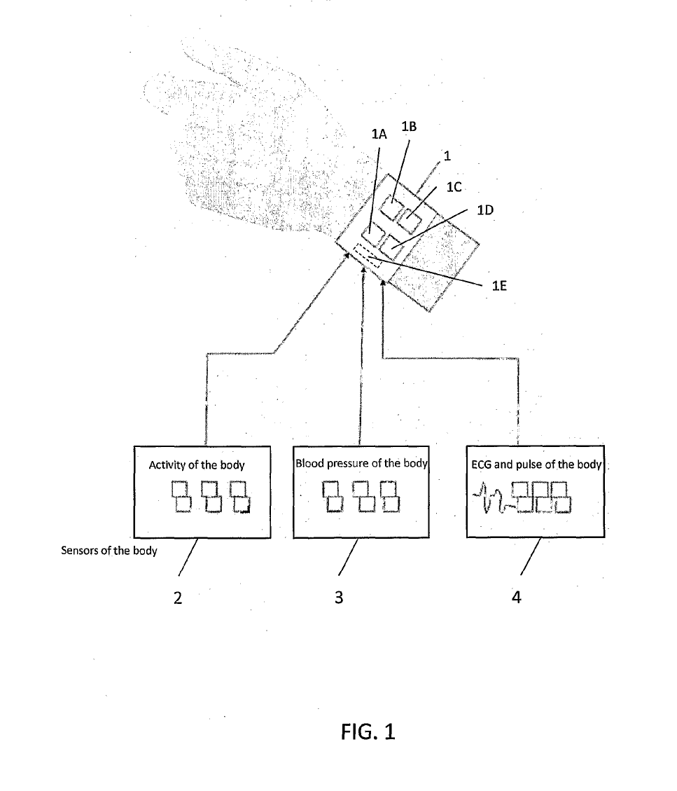

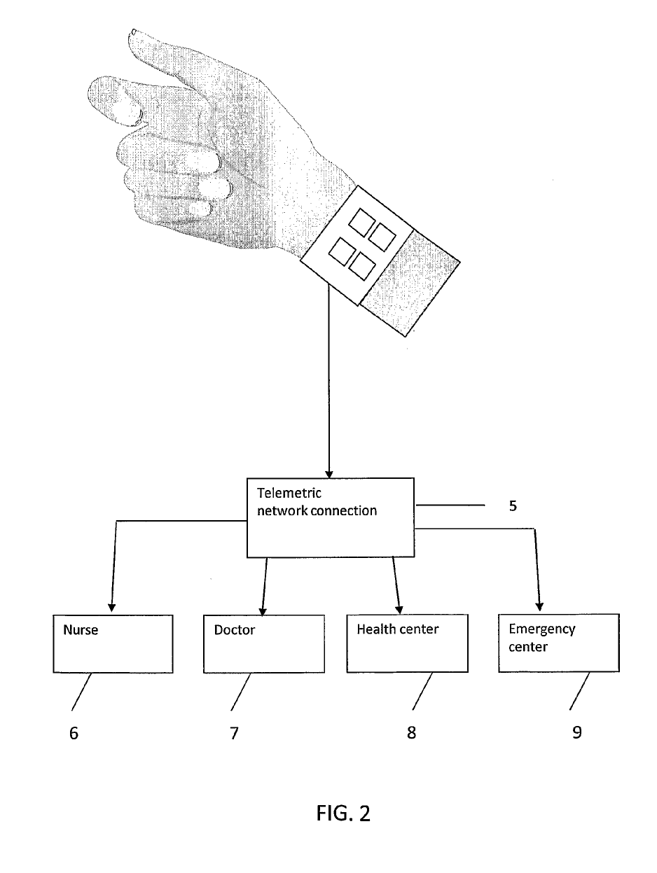

In the following the invention will be described in more detail with reference to the attached drawings, wherein FIG. 1 presents an apparatus according to the invention fixed the wrist of a person and also three different sensors. FIG. 2 presents an activity meter unit and three different transmitter units.

### DESCRIPTION OF PREFERRED EMBODIMENTS

FIG. 1 presents an apparatus according to the invention for wirelessly measuring an activity of a person. The apparatus according to the invention comprises an activity meter 1 on the wrist, to which sensors fixed to the body are wirelessly connected, an activity meter 2 of the body, a blood pressure gauge 3 of the body, and also an ECG and pulse counter 4 of the body. The activity meter 2 can comprise e.g. an acceleration sensor. The activity meter can comprise pushbuttons 1A-1D as well as a power source 1E.

The apparatus according to the invention operates as follows: a method and a device according to it measure an activity reading received from an activity sensor of the body, a blood pressure reading received from a blood pressure gauge of the body, and also an ECG curve and heart pulse reading received from sensors of the body. The measurement is continuous.

The apparatus according to the invention sends telemetrically an activity reading with a telemetric transmission unit 5 wirelessly to a server. According to one embodiment of the invention the activity data collected from a person can be recorded on the server. Sending of the data can be periodic. The server can process and analyze the data recorded in the server. After analysis of the measurement results the server converts the data into a suitable format for sending. The analyzed data can be delivered to users of the system in many formats, e.g. as an Internet page, an SMS message, an e-mail, or as another corresponding message, to a number of devices, e.g. to a mobile phone or to a computer. The measurement results from the server can be sent e.g. to a nurse 6, to a doctor 7, to a health center 8 and/or to an emergency center 9. This is presented in FIG. 2. The analyzed and recorded measurement results can also be available for the person being measured to use himself/herself and he/she can by means of them monitor his/her state of health and e.g. compare his/her own measurement results to the measurement results of a larger group formed of other users. The system can form a report, in which the condition of the person being measured is compared to the condition of a comparison group.

The apparatus of the invention can keep in contact with a server by means of a mobile phone network and e.g. TCP/IP protocol. For this purpose the apparatus can comprise a transmitter-receiver of a GSM, GPRS, 3G or other corresponding mobile phone system. Contact with a server can also be maintained by means of short-range radios such as Bluetooth or WLAN. In this case the system makes contact with a nearby base station, which directs messages to the server of the system.

The apparatus can take into account whether it is used in the domestic network of an operator or in the network of a foreign operator (roaming). In a domestic network the sending of data to a server can be performed e.g. more often than in the network of a foreign operator, so that the operating costs of the apparatus remain reasonable also outside the domestic network. The user can also define his/her own settings for the frequency of sending data. For example, in a foreign network the sending of measuring data can be completely forbidden.

The apparatus according to the invention can support bidirectional communications between the server of the system and the measuring apparatus. In this case the apparatus and its software can be updated from the server. This also enables remote management of the apparatus and the performing of settings e.g. by means of a web-based user interface.

The measuring apparatus of the system is designed such that the user can use it for long uninterrupted periods of time, so as to be able to offer continuous monitoring of the activity data of a person. In one embodiment of the invention the measuring device of the system is connected to one integrated device. The monitoring belt of the device can be manufactured from e.g. a flexible, cloth-like material.

The apparatus according to the invention is designed to operate for long uninterrupted periods of time and in this case the current supply of the apparatus must be sufficient. The batteries of the apparatus can be replaceable and/or they can be charged also during use of the device. The battery can be charged e.g. by means of an external power source or by means of an internal power source 1E that stores energy from the kinetic energy of a person. The charge and the need for charging of the battery can be presented to the user via the user interface of the apparatus.

The apparatus according to the invention comprises a memory, in which continuous measurement data can be recorded before they are sent to the server of the system. The measurement results for e.g. a period of several hours must fit in the memory because the measuring apparatus does not always necessarily make contact with the server or the connection to the server can be refused. When a connection to the server is in use, it is possible to e.g. send all the unsent measurement results in memory to the server.

The apparatus of the invention comprises an internal clock, so that it is possible to determine the time information of measurements of the device and the frequency of measuring. The clock of the apparatus can be set to the right time by means of e.g. a GSM network, a GPS system, some other external system or the own user interface of the device.

According to one embodiment of the invention the measuring apparatus does not comprise a separate display in which case the apparatus can be designed to be such in respect of its size and shape that it is easy to keep with oneself all the time. The device can comprise e.g. pushbuttons 1A-1D as a user interface. A number of users (e.g. 1-4 users) can use the same apparatus and the user can be selected with the pushbuttons 1A-1D. If the system uses mobile phone networks for data transfer, the apparatus comprises a location for a SIM card. Input of the PIN identification code of the SIM card can be set and automated e.g. in the commissioning stage of the apparatus, in which case a user interface for entering the code is not needed in the apparatus. In this case the apparatus can be kept simple and of small size.

When the apparatus is taken into use, a device is activated into the system. In the activation a device-specific, unique device identification and PIN code are created for the apparatus. The device identification and PIN code can be visible in the apparatus itself. The device identification or code can also be delivered in another manner, in which case they are not visible outside the device. The contact address or phone number of the server is stored in the apparatus. The contact information of the apparatus, e.g. the phone number of the apparatus, for bidirectional data transfer is also registered in the server. In addition to this, a user identification can be created in the system for a user of the apparatus. By means of activation use of the apparatus can, if desired, be prevented, e.g. owing to unpaid payments of the user, and in this case the measuring apparatus can be extinguished.

It is obvious to the person skilled in the art that the different embodiments of the invention are not limited solely to the embodiment described above, but that they may be varied within the scope of the claims presented below.

1. Method for measuring the activity of the functions of the body of a person, wherein the vital functions are measured with a portable activity measuring apparatus (1-4), characterized in that
an activity of the body is measured wirelessly such that a sensor or sensors, e.g. activity sensors, blood pressure sensors, ECG sensors or heart pulse sensors, that are fixable to the body are used for the measuring of the activity, from which sensor or sensors a measured value or measured values are received, e.g. activity values, blood pressure values, ECG curves or heart pulse values,
the aforementioned value or aforementioned values are sent wirelessly to the server of the measuring of an activity for processing, monitoring, analysis and/or other actions, and
the server analyzes the measured values, converts the processed measurement results into a suitable format for sending onwards and sends the converted measurement results to the user/users.

2. Method according to claim 1, characterized in that the measured values are measured with at least two different types of sensors.

3. Method according to claim 1, characterized in that the processed measurement results are sent from the server to experts, such as nurses, doctors and health centers and/or to emergency centers, for actions.

4. Method according to claim 1, characterized in that the processed measurement results are sent from the server back to the activity measuring apparatus.

5. Method according to claim 1, characterized in that the measurement occurs continuously.

6. Method according to claim 1, characterized in that in the server own measurement results can be compared to the measurement results of a larger group.

7. System for measuring the activity of the functions of the body of a person, which system comprises a portable activity measuring apparatus and a server of the measurement of an activity, characterized in that
the activity measuring apparatus is fitted to measure an activity of the body wirelessly such that the apparatus comprises a sensor or sensors, e.g. activity sensors, blood pressure sensors, ECG sensors or heart pulse sensors, that are fixable to the body are used for the measuring of the activity, from which sensor or sensors a measured value or measured values are received, e.g. activity values, blood pressure values, ECG curves or heart pulse values,
the activity measuring apparatus is fitted to send the aforementioned measured value or aforementioned measured values to the server of the measuring of an activity for monitoring, analysis and/or other actions and
the server is fitted to analyze the measured values, to convert the processed measurement results into a suitable format for sending onwards and to send the converted measurement results to the user/users.

8. System according to claim 7, characterized in that at least two different types of sensor are in use in the activity measuring apparatus.

9. System according to claim 7, characterized in that the server is fitted to send the processed measurement results wirelessly to experts, such as nurses, doctors and health centers and/or to emergency centers, for actions.

10. System according to claim 7, characterized in that the activity measuring apparatus comprises an activity meter (1), which comprises a display and a transmitter part and which is fixed to the arm.

11. System according to claim 7, characterized in that the activity measuring apparatus comprises a power source of the automatic measuring device of a human body, in which power source charging is arranged to operate when kept on the wrist of a person and stored with the kinetic energy of the wrist movement of the person.

12. System according to claim 11, characterized in that the power source is separate and the voltage received from the power source can be transferred to an activity meter that is fixed to the arm.

13. System according to claim 11, characterized in that the power source is integrated into an activity meter that is fixed to the arm.

14. System according to claim 7, characterized in that the activity meter is fitted to telemetrically send an activity reading with a telemetric transmission unit (5).

15. System according to claim 9, characterized in that the activity meter is fitted to telemetrically send an activity reading with a telemetric transmission unit (5).

16. System according to claim 10, characterized in that the activity meter is fitted to telemetrically send an activity reading with a telemetric transmission unit (5).
